(19) United States
(12) Patent Application Publication
(10) Pub. No.: US 2026/0041539 A1
(43) Pub. Date: Feb. 12, 2026

(54) BIONIC INTERACTIVE SPHINCTER/SLING WITH FLOATING VARIABLE CIRCUMFERENCE

(71) Applicant: Peter H. Sayet, Fort Lauderdale, FL (US)

(72) Inventor: Peter H. Sayet, Fort Lauderdale, FL (US)

(21) Appl. No.: 18/798,248

(22) Filed: Aug. 8, 2024

Related U.S. Application Data
(60) Provisional application No. 63/471,915, filed on Jun. 8, 2023.

(51) Int. Cl. A61F 2/00

(52) U.S. Cl. CPC A61F 2/0036; A61F 2220/0033; A61F 2250/001; A61F 2250/0037; A61F 2250/0073

(57) ABSTRACT

An implantable system for compressing internal tissue within a host body includes a compressing member and a stricture member made up of several interactive components for compressing internal tissue that could include a body canal which is a conduit for the flow of internal or external materials including fluids or solids or just a targeted tissue mass within the host body. The compressing aspect, including the stricture member, can also act to alternatively release said compression on the targeted tissue. In creating compression, the compressing aspect moves against the stricture member while reversing the compression is accomplished by reversing the process. An actuator, controlled by internal or remote electronic signals acts to move the compressing member.

This Utility Application claims the priority of the Provisional U.S. Application No. 63/471,915, filed Jun. 8, 2023, under 35 U.S.C. 119(e). Applicant also respectfully submits a Petition under 37 CFR 1.78(a)(1) to restore and claim the benefit of the above Provisional Patent Application. The delay in filing the subsequent nonprovisional application within the twelve-month period set forth in Paragraph 37 CFR 1.78(a)(1) was unintentional.

### FIELD OF THE INVENTION

The invention relates to implantable medical devices that can compress a variety of tissue masses within a host body wherein compressing such tissue can result in the treatment of various medical conditions.

### BACKGROUND OF THE INVENTION

The plumbing systems of most living creatures created by nature over the last 500 million years are among the most essential functions of those creatures with the plumbing system of the current human among the most sophisticated of all. The plumbing systems embodied in vertebrae animals (including humans) perform multiple functions including managing the flow of internal and external materials spanning liquids (i.e.: blood and urine, and the like) to solids (feces created from the ingestion of foreign food or otherwise material) and even air or other cellular material. Within these plumbing systems there exists a number of different involuntary and voluntary valves or aperture open/closing mechanisms or structures, including muscle groups known as sphincters.

Like everything else in the animal anatomy, all the structures that are included in the animal's plumbing systems are subject to losing proper function as a result of aging and/or disease or just as a result of the side effect of non-related disease processes, which can greatly impact the health and/or well-being of the host animal (human).

As an example, the act of childbirth in females can cause a fundamental change in the lower abdominal anatomy of the birther by stretching or repositioning, often on a permanent basis, various tissue and muscles in the region including those muscles (sphincters) that are vital to maintaining both urinary and fecal continence.

Of course, there are many other conditions and disease states that can cause both males and females to become (either or both urinary and fecal) incontinent including, but not limited to; neurogenic diseases, autoimmune diseases, spinal cord injuries, serious gut disorders and even dementia and Alzheimer's.

Accordingly, in urinary and fecal incontinence (UI, FI), the onset of problems and issues with every component of the lower urinary and fecal tracts can result in incontinence.

Among the most common problems is the reduction or loss of function of a person's urinary or fecal sphincters.

Given the primary function of the urinary and fecal sphincters is to simply open or close on the requisite tubular organ (the urethra or rectum, respectively), it is expected the primary way to regain continence is some sort of device that can, as close as possible, duplicate the open/close compression/decompression action of the sphincters.

Although the open/close action of candidate devices is rather straight forward, the true challenge for such (foreign material) devices is to do so without causing an unacceptable (the least) amount of damage to the tissue or the patient.

The invention envisioned herein has been designed to achieve in a unique way the open/close function at a high degree of efficacy while avoiding causing an unacceptable amount of damage to the tissue and patient over extended timespans.

There are a number of medical conditions, including urinary and fecal incontinence, GERDs, morbid obesity, Hyperhidrosis, post-ostomy surgery control of waste material, nerve and pain disorders and even cognitive or physical disabilities that could be treated using some means of compressing and decompressing internal tissue in a host body. These conditions can arise from various causes. The causes include a broad variety of related and unrelated disease conditions, injuries, aging and the general loss of functionality of some aging tissue, host induced health issues and the side effects of other treatments. The costs of these conditions to both individuals and worldwide healthcare systems are substantial. Several treatments currently exist that are known in the prior art. Among these are surgical corrections (minor and major), drugs, and in the case of both urinary and fecal incontinence, devices, or more properly stated, apparatuses that capture or contain host waste discharges (i.e.: diapers and catheter systems while ostomy bag systems are used for post-ostomy surgery patients). Given the broad range of prospective uses of the subject invention, other treatment solutions can vary quite considerably from a variety of drugs, surgical procedures to artificial sphincters, slings and neuromodulation devices for incontinence to bands or sleeves and neuromodulation systems for morbid obesity to drugs and a device for GERDs and to drugs and neuromodulation systems for pain and nerve disorders. Multiple safety and efficacy issues including a substantial list of adverse events associated with each of the currently in-use treatments continue to significantly affect the use of all the current treatment modes especially amongst those afflicted people who exhibit the more serious cases, making it essential to continue to invent and incorporate newer, prospectively more efficacious, and safer treatment modes.

### SUMMARY OF THE INVENTION

The present invention overcomes a number of the efficacy and safety issues that are associated with all the currently in-use treatment modes in each of the treatment areas cited above including eliminating many of the adverse events or reducing their severity while improving efficacy at costs that could result in a reduction of the expenses currently being expended.

Generally speaking, and in accordance with the invention, an implantable mechanism for compressing a variety of tissue masses in a host body comprises (1) a stricture member for reducing the circumference of the tissue mass when in the compressed position while alternatively allowing the tissue mass to regain its natural static form when the stricture member is in its decompress position, (2) a compressing member that moves against the stricture member to cause the stricture member to reduce the circumference of the tissue mass being compressed while alternatively allowing the tissue mass to regain its natural static form when the compressing member is retracted off the stricture member to its decompress position, (3) a moving member connected to the compressing member that alternatively moves toward or away from the surrounded tissue mass, and (4) an actuation member as controlled by internal or remote electronic signals acts to move the moving member.

Among the most pressing concerns for any implanted device that is designed to compress the internal tissue of host bodies is the level of damage that is caused to the tissue from such compression especially with use over protracted periods of time.

The present invention incorporates multiple new design features that are directly related to the best possible preservation of the tissue it is compressing and are key to the present invention's superior features as compared to all the currently in use prior art treatment modes across all the treatment options available for all the medical conditions cited with the expectation that efficacy rates can be improved, and safety issued substantively reduced.

Although sharing some common design features with other devices in the treatment spaces cited which design features have long been in use (i.e.: devices that surround and compress tissue masses, and the like), the present invention departs significantly with the key design features of all the prior art known for the way and means it acts on the surrounded tissue mass making its design unique.

The drawings and specific descriptions of the drawings, as well as any specific or alternative embodiments discussed, are intended to be read in conjunction with the entirety of this disclosure. The invention may be embodied in many different forms and should not be construed as being limited to the embodiments set forth herein; rather, these embodiments are provided by way of illustration only and so that this disclosure will be thorough, complete and fully convey understanding to those skilled in the art. The above and yet other objects and advantages of the present invention will become apparent from the hereinafter set forth Brief Description of the Drawings, Detailed Description of the Invention, and claims appended herewith.

### BRIEF DESCRIPTION OF THE DRAWINGS

The foregoing and other objects, advantages and features of the present invention, and the manner in which the same are accomplished, will become more readily apparent on consideration of the following detailed description of the present invention taken in conjunction with the accompanying FIGS which illustrate preferred and exemplary embodiments and wherein:

FIG. 1 is an isometric side view of a bionic interactive sphincter/sling with floating variable circumference device according to the invention as seen in the pre-implant pre-engaged position.

FIG. 2 is a cross-sectional view of the valve/cuff assembly of a bionic interactive sphincter/sling with floating variable circumference device according to the invention as seen in the pre-implanted disengaged position.

FIG. 3A is an isometric side view of the whole device embodiment of a bionic interactive sphincter/sling with floating variable circumference device according to the invention as seen in the full-opened position after engaging the locking mechanisms.

FIG. 3B is a cross-sectional view of the valve/cuff assembly of a bionic interactive sphincter/sling with floating variable circumference device according to the invention as seen in the full-opened position after engaging the locking mechanisms.

FIG. 4A an isometric side view of the whole device embodiment of a bionic interactive sphincter/sling with floating variable circumference device according to the invention as seen in a partially closed position after engaging the locking mechanisms.

FIG. 4B is a cross-sectional view of the valve/cuff assembly of a bionic interactive sphincter/sling with floating variable circumference device according to the invention as seen in a partially closed position after engaging the locking mechanisms.

FIGS. 5A and 5B are illustrations of an alternative embodiment of the inner flexible band wherein it is flared at the midsection where the valve/actuator member impacts it when moving to and sitting in the closed position.

### DETAILED DESCRIPTION OF THE INVENTION

The invention herein provides a solution for bionic sphincter control. The invention includes a uniquely configured strap that is actuated by a motor to constrict or release as needed capable of solving the above issues.

Previous means of compressing an internal vessel or tubular organ like a urethra or a rectum included using a cuff with expandable walls, like a balloon or blood pressure cuff, that surrounds the vessel which could be filled and emptied of some fluid that would in turn expand or deflate the cuff via some form of triggering or activation mechanism that controlled a valve placed near a fluid reservoir for access and egress of the fluid in and out of the reservoir and cuff per the activator.

Another means of compressing an internal vessel or tubular organ like a urethra included a plunger attached by a wire cable to a stepper motor that could move on and off the targeted vessel to alternatively compress or decompress the vessel via the use of an activation mechanism.

A third means of compressing an internal vessel or tubular organ like a urethra included the use of some material, either synthetic or biological, that is positioned like a sling or hammock so that it pulls against the vessel causing a partial closure or obstruction of said vessel.

In the cases of stationary non-dynamic treatments such as pessaries, and even some synthetic or biological materials used as bulking agents, all these and other devices have been used to partially obstruct a vessel or tubal organ, or in the case of devices capable of moving on and off the vessel, partially or fully obstruct and then un-obstruct the targeted vessel or tubal organ, especially the human urethra (and rectum), in an effort to treat certain medical conditions including urinary and fecal incontinence (UI, FI).

Accordingly, the use of compression devices or bulking materials are among the most common treatment modes for human conditions wherein human sphincter muscles are not working optimally.

Notwithstanding, the key to creating an effective substitute or assist artificial sphincter is whether or not it can perform effectively while still not causing unacceptable levels of damage to the tissue it's compressing as a result of the action of compressing internal tissue, which is the main weak spot for all the current array of treatment choices.

As such, new artificial sphincter/valve technologies have to continue to advance the efficacy in balance with better preservation of the tissue.

The Bionic Interactive Sphincter/Sling (BISS) with floating variable circumference or Variable Circumference Flow Control Valve Collar is just such a new technology.

The BISS design incorporates several features that are unique including incorporating a unique flexible inner band to the mechanism that allows the activator to actively but gently disperse the pressure on the vessel or tubal organ over a much larger section of the vessel than a plunger alone.

This addition of a flexible inner band to the flexible outer belt/strap anvil will effectively act like a variable circumference cuff which only impacts a reduced section of the circumference of the vessel or tubal organ it is compressing while substantively reducing the impact the device has on the compressed tissue increasing the efficacy but also reducing the damage caused to the compressed tissue.

This combination of inner and outer flexible bands will also allow the entire cuff, when open, to completely disengage off all contact points on the targeted tissue and thereby float along the available length of the targeted vessel or tubal organ reengaging with the vessel at prospectively various different contact points when moved to the closed position.

This ability to float along the available length of the vessel allows the cuff to engage the vessel at possibly slightly different positions avoiding compressing the vessel at precisely the same contact points each time as a means of mitigating the damage that can be caused by compressing the vessel on the precisely the same contact points over the course of a protracted number of activations.

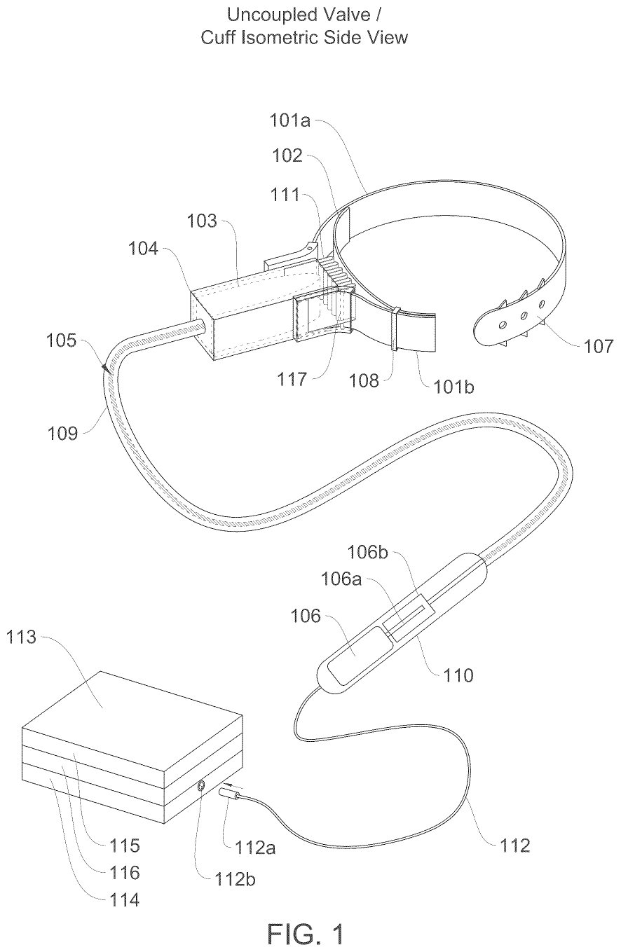

The construction of this device may be appreciated in FIG. 1. An outer anvil belt/strap 101a/b attaches permanently on one side of the engaging housing 101a while the distal end has a belt-like structure 107 with several sizing holes or flanges that can marry up with a locking mechanism like a belt buckle which is permanently attached to the opposite of the engaging housing from the anvil belt/strap 101a/b to provide the appropriate maximum circumference setting of the head unit anvil for each vessel or tube or tissue mass it is surrounding and compressing.

An inner flexible band 102 is attached either singularly at the distal end or both at the proximal and distal ends to the inside wall of the outer anvil belt/strap 107/108 allowing the full length of the inner flexible band to flex (inward) when pushed against by the actuator engaging 103

All implanted parts that come in contact with any internal tissue or which are exposed to any internal environment of the host human or animal body will be physically made from inert materials that have previously been used and have been found safe for long-term use internally in human or animal bodies. The two outer anvil strap/belt pieces 101a and 101b and inner flexible band 102 piece will be constructed of materials that are flexible enough to bend sufficiently during the implant procedure around any internal body tissue mass while also being supple and soft enough not to cause an unacceptable level of damage to the surrounded tissue mass over protracted timespans of compression and may be cushioned to do so. The two outer anvil strap/belt pieces will have accompanying hinges 117 affixed to their proximal ends that are attached to the valve/actuator housing 104 along with accompanying limiters to contain the flex angles of the anvil strap/belts 101a/b while also helping to avoid causing any inadvertent compression on the targeted tissue by these pieces. The valve/actuator housing 104, motor housing 110 and electronics casing 113 will be constructed of materials of sufficient solidity and rigidity to maintain their physical shapes during long-term exposure to the entire internal body environment in which they will be deployed while also being air and fluid tight along with their seals to prevent any incursion of air or fluids into the housing. The valve/actuator seal 111 will be constructed of a balloon-like material while the valve/actuator 103 itself could be structured and constructed of a range of materials that could go from a more solid piece all the way to a somewhat rigid, but more balloon-like air or fluid filled structure. The motor push wire 105 could be constructed of a variety of materials that have the property of being strong in tensile strength but flexible, from a metallic to a plastic compound while the motor push wire sheath 109 will be constructed of a flexible plastic that will closely match the flexibility of the motor push wire 105. The electronic wire 112 will be a standard electronics wire that will either be coated or will be covered by a plastic sheath 109 and will have a male jack plug 112b affixed to its distal end away from the motor 110. The male locking mechanism 107 will be an extension of the distal end of the outer anvil/strap 101a piece that could have several spaced outer edges or grooves (not labeled in illustration) and be constructed to fit into the female locking mechanism 108 of the 101b anvil strap/belt piece that could be made of a more rigid material and may include associated locking pins (not labeled in illustration). The PC board 114 and power supply 116 will be encased in the electronics case 113 while both the telemetry 115 and battery recharger (not shown in illustration) conduits will be connected to the power supply and may or may not protrude out of the electronics case 113 and may be covered by a plastic subcase or further encased in a resin that does not interfere with electronic signals. The electronics case 113 will hermetically seal off any of its internal components from ever coming in contact with any tissue or internal environment of the host body. The electronic case will be connected to the motor assembly via its on-board jack receptacle 112b that will be a plug-in/plug-out facility for the male jack end 112a of the connecting wires 112 of the motor.

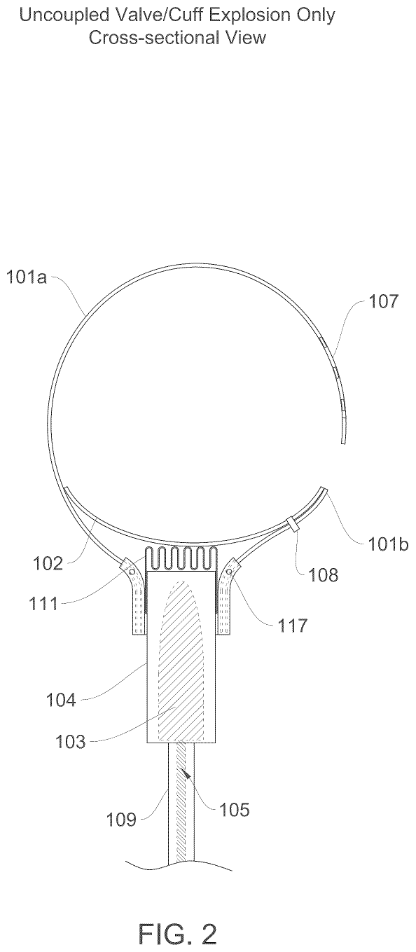

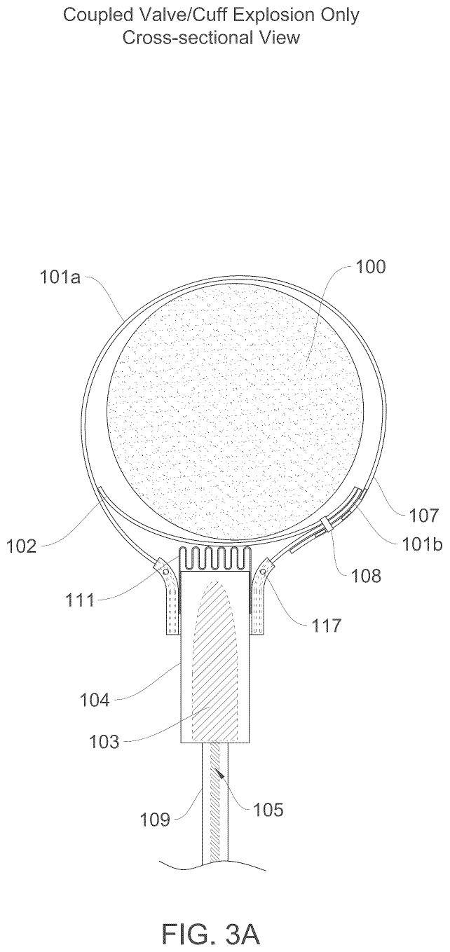

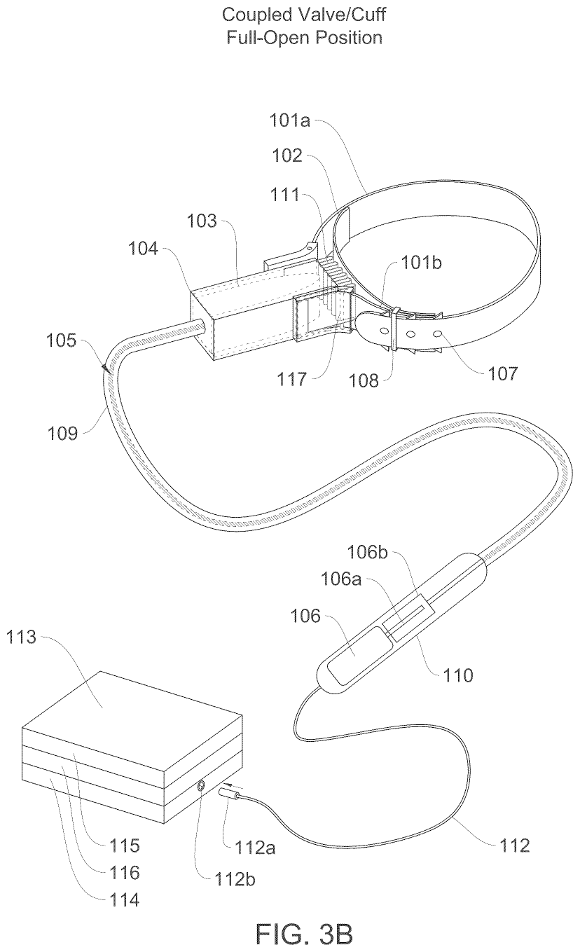

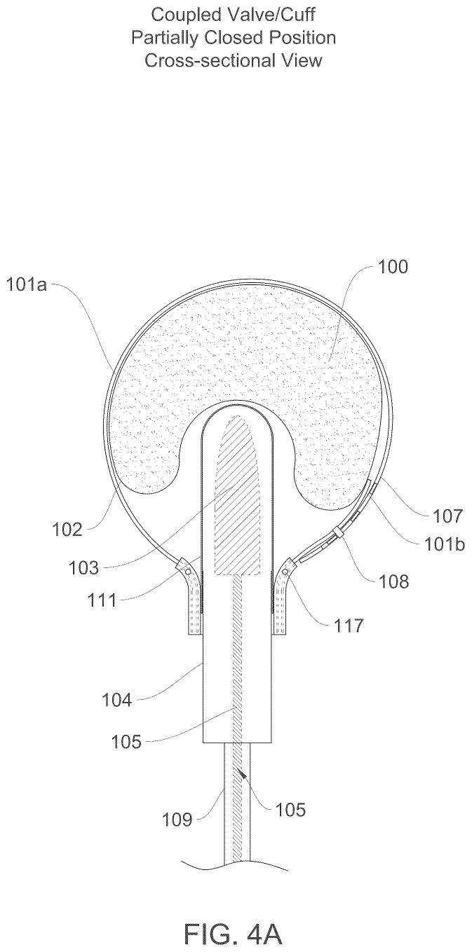

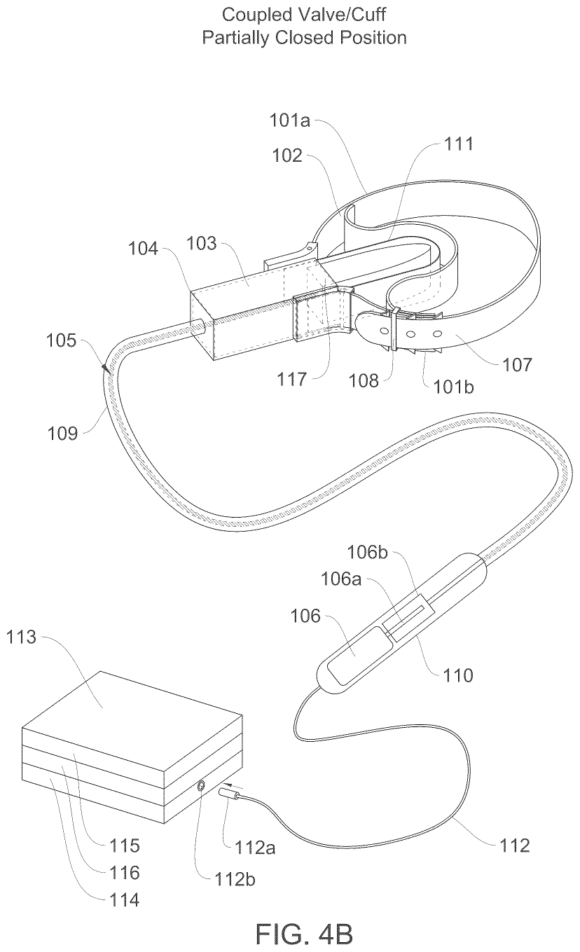

Referring now in more detail of the FIGS in which like numerals refer to like parts throughout several views, FIG. 1 shows a first partial side explosive isometric view as the device appears prior to it being engaged in the implanted position with FIG. 2 showing a cross-sectional view of the valve/cuff assembly as seen in the pre-implant pre-engaged position while FIG. 3A including urethra 100 and FIG. 3B show an additional side isometric view of the full device embodiment and the associated cross-sectional view of only the valve/cuff assembly, respectively, of the device in the full-open position followed by FIG. 4A and FIG. 4B a yet another additional side isometric view of the full device embodiment and the associated cross-sectional view of only the valve/cuff assembly, respectively, of the device in one of its partially closed positions after the bionic interactive sphincter device is engaged according to the present invention as it might be positioned around a tissue mass of the host. In FIG. 1 as the preferred embodiment, the bionic interactive sphincter/sling device comprises a first engaging including parts 101a and 101b with 101a the longer of the two anvil strap/belt pieces being anchored at the proximal end (proximal to the actuator housing 104) via the anvil strap/belt hinge 117 to one of the flat sides of the actuator housing 104 and have at its distal end the male locking mechanism 107 while the second shorter piece will be anchored at its proximal end (proximal to the actuator housing 104) via the anvil strap/belt hinge 117 to the other of the flat sides of the actuator housing 104 (opposite to where the longer first engaging 101a is anchored) and will have at its distal end the female locking mechanism 108, with a second engaging 102 and a third engaging 103 that includes a housing 104 and a seal 111. The two free ends of the second engaging 102 are already affixed to the inside surfaces of the two pieces of the first engaging (101a and 101b) which are fully affixed to the housing 104 of the third engaging 103 and when the male 107 and female 108 locking mechanisms are coupled, an inner diameter is formed which is well suited for fitting around a host body canal or any internal body tissue mass (i.e., any tube, canal or tissue mass within the human or other animal body such as the urethra 100 or rectum or stomach or nerve bundle). The bionic interactive sphincter device also includes a sizing and male locking mechanism 107 on the free side distal end of the first engaging 101a for varying the size of the inside circumference when it is engaged and coupled to the female locking 108 on the distal end of the 101b piece further including the second 102 engaging in the sizing of the circumference with both pieces 101a and 101b attached to the housing member 104 of the third engaging 103. Any other equivalent locking mechanism could be used for this purpose. Alternative locking mechanisms contemplated by the present invention include but are not limited to, the use of a strap and snap pins or interconnected moldings on the male 107 and female 108 locking mechanisms. The bionic interactive sphincter devices of the present invention further include a valve/actuator member 103 and an inner flexible band 102 within the outer band diameter formed by the coupling of the male 107 and female 108 locking mechanisms such that the valve/actuator member applies pressure to the inner band 102 that will reduce the inner circumference of the present invention and thereby press against the surrounded targeted tissue causing the tissue to compress. It should be noted that if the targeted tissue mass is some form of a vessel, tube or even organ that has an internal cavity or tunnel space, the reduction of the circumference of the targeted tissue mass will necessarily reduce the inner space of the targeted tissue mass.

A biasing member is utilized by a bionic interactive sphincter/sling device of the present invention to force the valve/actuator 103 to normally apply a compressive force on the inner flexible band 102 which in turn will apply a compressive force on the tissue mass when the bionic interactive sphincter/sling device is in the closed or closing position. The same drive force deployed by the bionic interactive sphincter that moves the valve/actuator 103 to apply a compressive force on the inner flexible band 102 can be reversed to effectuate the decompression on the inner flexible band 102 and thereby also decompressing the targeted tissue mass. The compression/decompression member comprises a motor push wire 105 covered by a push wire sheath 109 with a proximal and distal end (proximal end to the actuator member 103) with its connection at the proximal end to the actuator member 103 and to the floating drive/stepper motor 106 at the distal end while the push wire sheath 109 is affixed at its proximal end to the actuator housing 104 while the distal end is affixed to the motor housing 110 which allows the motor push wire 105 to slide freely within the push wire sheath 109 based on the moving forces generated by the stepper motor 106 and consequently also transferring that motion to the valve/actuator member 103 and the inner band 102. The push wire sheath 109 is designed to hermetically seal and protect the push wire 105 from coming in contact with any tissue or internal host body fluids.

FIG. 2 illustrates the valve/actuator member 103 in the cross-sectional view of the valve/cuff assembly only in the pre-implant disengaged position while FIG. 3B and FIG. 4B illustrate the valve/actuator member 103 in the same cross-sectional view of the valve/cuff assembly only, once the two anvil strap/belt pieces are coupled around the targeted tissue via their locking mechanisms in the full-opened position and alternatively, in one of the different closed positions, respectively, which closed positions could vary based on the telemetry signals being sent by the associated software.

The stepper motor 106 assembly includes a motor, gearbox, threaded shaft 106a and a cable mounting structure 106b that allows the floating drive/stepper motor 106 to transfer its rotating energy to a perpendicular movement that results in the opening and closing movement that is the basis of the operations of the bionic interactive sphincter/sling devices.

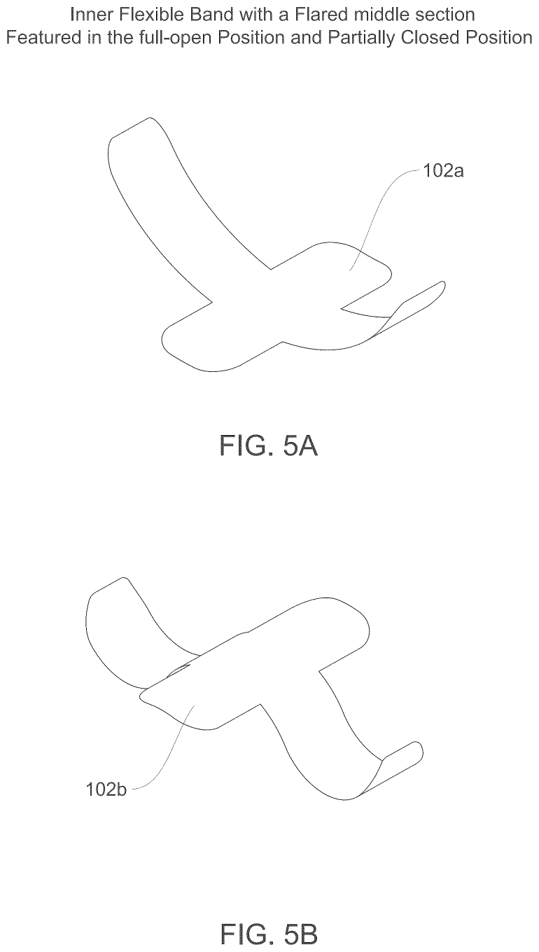

FIGS. 5a and 5b are alternative embodiments of the inner band 102a and 102b wherein it can be designed to flair out in its midsection where it is impacted by the valve/actuator member 103 spreading its contact pad against the targeted tissue over a longer and wider footprint to provide more closing compression on the targeted tissue with a reduced pressure per square surface size.

The means by which the inner band 102a and 102b diffuse the compression exerted on the tissue mass when it's in the closed position is what makes the bionic interactive sphincter/sling device unique. Instead of the actuator 103 directly impacting on the tissue mass, the actuator mechanism for the bionic interactive sphincter device/sling impacts the inner band 102a and 102b which then diffuses that impact on the tissue mass over a much broader contact pad thereby creating less opportunity for compression damage caused to the targeted tissue mass.

Accordingly, it will be understood that various embodiments of the present invention have been disclosed by way of example and that other modifications and alterations and may occur to those skilled in the art without departing from the scope and spirit of the claims. Thus, the invention described herein extends to all such modifications and variations as will be apparent to reader skilled in the art, and also extends to combinations and sub-combinations of the features of this description and the accompanying FIGS. and described in the foregoing detailed descriptions it will be understood that the present invention is not limited to the embodiments disclosed, but is capable of numerous rearrangements, modifications and substitutions without departing from the spirit of the present invention as set forth and defined by the following claims, such as for example those embodiments described in non-provisional U.S. Pat. Nos. 6,319,191 B1 and 6,527,701 B1, which are incorporated hereinto in their entirety by reference.

While there has been shown and described above the preferred embodiment of the instant invention it is to be appreciated that the invention may be embodied otherwise than is herein specifically shown and described and that certain changes may be made in the form and arrangement of parts without departing from the underlying ideas or principles of this invention as set forth in the Claims appended herein.

I claim:

1. An implantable apparatus to compress internal tissue masses within a host body comprising:
a compressing or biasing member for compressing a tissue mass when in the closed position; said compressing member comprising an actuator member applying pressure to a stricture member for applying pressure to an internal tissue mass; and
a pulling member to reverse the compression put on the actuator member and structure member such that when activated, it will force the activator member to move away from the closed position and allow the targeted tissue mass to return to its original static form with the activator member being mechanically connected to the pulling member.

2. The implantable apparatus to compress internal tissue masses within a host body, as recited in claim 1 further comprising:
a constricting member, wherein said constricting member is a belt-like strap that consists of two separate parts, with the first part attached to one of the flat sides of the actuator housing at its proximal end, wherein said proximal end is proximal to the actuator housing, while the distal end is formed into a male designed counterpart of a locking mechanism that has a male and female counterpart.

3. The implantable apparatus to compress internal tissue masses within a host body, as recited in claim 1 further comprising:
a constricting member, wherein said constricting member is a belt-like strap that consists of two separate parts, with a second part attached to an opposite flat side to a side whereon the first part is attached to an actuator housing while a distal end is formed into a female designed counterpart of a locking mechanism that has a male and female counterpart.

4. The implantable apparatus to compress internal tissue masses within a host body, as recited in claim 1, wherein the locking mechanism includes at least selected from a group, including:
sliding the male counterpart piece into and through the female counterpart piece to be positioned over raised locking protrusions on the surface of the female piece, wherein the male piece has grooved edges or notches that can slip through a bar affixed to the female counterpart piece wherein the anvil belt/strap will adjust its resting diameter at one of several available notch/groove positions;

5. The implantable apparatus to compress internal tissue masses within a host body, as recited in claim 1, wherein an inner flexible band constricting member is a strip of material that may be of a different width side-to-side of the anvil belt/strap and could vary in length as compared to the anvil belt/strap and is affixed at both the proximal and distal ends to the inside of the anvil belt/strap with one end affixed to the male counterpart and the other affixed to the female counterpart of the anvil belt/strap.

6. The implantable apparatus to compress internal tissue masses within a host body, as recited in claim 1, wherein an inner flexible band constricting member is also attached to the valve/actuator or the actuator seal on its outside side such that when the valve/actuator moves toward the surrounded tissue mass it pushes the inner flexible band against the tissue mass and thereby compressing it while when the valve/actuator moves away from the inner flexible band it thereby pulls the inner flexible band off the tissue mass allowing the tissue mass to resume taking its more natural decompressed form.
wherein the use of an inner flexible band helps to distribute the closing force on the surrounded tissue over a much larger contact point than what would be the case with the valve/actuator member directly impacting the surrounded tissue and would consequently complete a compression sequence that would more evenly constrict the surrounded tissue but do it with a less focused force thereby doing less damage to the surrounded tissue during the compression action.

7. The implantable apparatus, as recited in claim 1, to compress internal tissue masses within a host body, further comprising:
a head unit comprised of the inner and outer flexible band and anvil belt, and the valve/actuator and housing are designed to fully disengage from being in contact with whichever tissue mass it is surrounding to compress as a means of allowing the tissue mass to being exposed to the surrounding natural bodily environment while the device is in the open position which further allows for the device to reengage the tissue mass while moving to the closed position at a slightly different pressure contact point on the tissue mass thereby creating a means of avoiding tissue damage to a specific contact patch on the tissue mass.

8. The implantable apparatus, as recited in claim 1, to compress internal tissue masses within a host body, further comprising:
a drive/motor assembly is designed to “float” within any nearby bodily cavity to the site of the placement of the valve/actuator and its housing by virtue of having a flexible actuator wire/cable that is some length away from the valve/actuator while also being connected to the present invention's power supply and computer control chip via a flexible electronic wire/cable that is some length away from the drive/motor system with the power supply/PC-board control chip assembly preferably positioned in some subcutaneous space nearby a device head assembly and in concert results in suspending the drive/motor assembly in the cavity; and
wherein said device head assembly includes a valve/actuator, retaining belt/strap and inner band subassembly.

9. The implantable apparatus, as recited in claim 1, to compress internal tissue masses within a host body, further comprising:
a drive/motor assembly that is in one possible configuration not part of the subcutaneous control pack assembly allowing the control pack assembly to be significantly reduced in size resulting in a better cosmetic appearance with less disruption to the subject user and avoidance of having to change out the drive components earlier than their useful lives because of other adjacent component failures.

10. The implantable apparatus, as recited in claim 1, to compress internal tissue masses within a host body, further comprising:
a PC-board assembly that is in one possible configuration not part of the subcutaneous control pack assembly allowing the control pack assembly to be significantly reduced in size resulting in a better cosmetic appearance with less disruption to the subject user and avoidance of having to change out the PC-board component earlier than its useful life because of other adjacent component failures.

11. The implantable apparatus, as recited in claim 1, to compress internal tissue masses within a host body, further comprising:
a power supply assembly that is in one possible configuration not part of the subcutaneous control pack assembly allowing the control pack assembly to be significantly reduced in size resulting in a better cosmetic appearance with less disruption to the subject user and avoidance of having to change out the power supply component earlier than its useful life because of other adjacent component failures.

12. The apparatus as recited in claim 1, comprising;
the anvil belt/strap hinges, which may also have limiters, have been incorporated to control the flex angle of the two-anvil belt/strap pieces to allow for easier placement while helping to avoid inadvertent compression on the targeted tissue.
